(19) United States
(12) Patent Application Publication
(10) Pub. No.: US 2018/0020969 A1
(43) Pub. Date: Jan. 25, 2018

(54) SYSTEM AND METHOD FOR CONTROLLING THE CYCLIC MOTION OF A BODY SEGMENT OF AN INDIVIDUAL

(71) Applicants: UNIVERSITE GRENOBLE ALPES, St Martin D'Heres (FR); CENTRE HOSPITALIER UNIVERSITAIRE GRENOBLE, La Tronche (FR); CENTRE NATIONAL DE LA RECHERCHE SCIENTIFIQUE (CNRS), Paris (FR)

(72) Inventors: Philippe CINQUIN, St. Nazaire Les Eymes (FR); Vincent NOUGIER, Saint Ismier (FR); Jacques GRIFFET, Meylan (FR); Aurélien COURVOISIER, Grenoble (FR); Lucas STRUBER, Grenoble (FR); Jean DUBOUSSET, Paris (FR)

(73) Assignees: UNIVERSITE GRENOBLE ALPES, St. Martin d'Hères (FR); CENTRE HOSPITALIER UNIVERSITAIRE GRENOBLE, La Tronche (FR); CENTRE NATIONAL DE LA RECHERCHE SCIENTIFIQUE (CNRS), Paris (FR)

(21) Appl. No.: 15/536,495

(22) PCT Filed: Dec. 18, 2015
(86) PCT No.: PCT/FR2015/053653

§ 371 Date: Jun. 15, 2017

(30) Foreign Application Priority Data
Dec. 18, 2014 (FR) 1462735

(51) Int. Cl. A61B 5/00; A61B 5/11

(52) U.S. Cl. CPC A61B 5/486; A61B 5/6804; A61B 5/0024; A61B 5/7405; A61B 5/742; A61B 5/7455; A61B 5/1116; A61B 2505/09; A61B 2560/0223; A61B 2562/0219

(57) ABSTRACT

A system controls a cyclic motion of at least one body segment of an individual, including:
at least one three-dimensional motion sensor,
an attachment attaching the sensor to the body segment of the individual,
a processor coupled to the sensor and capable of being carried by the individual, in which a reference cyclic motion and tolerance limits specific to the individual concerning at least one parameter of the motion to be controlled, which change depending on the progress thereof, are stored, the processor being configured to detect the cyclic motion, integrate the measurement data from the sensor in such a way as to calculate the parameter(s) of the motion of the segment and to compare, in real time, the parameter(s) of the measured motion (Cₜᵣ) with the parameter(s) of the previously recorded reference motion (C_{ref}) of the individual, and
a feedback device coupled to the processor, designed to provide the individual with real-time information on the compliance of the parameters(s) of the measured motion with respect to the parameter(s) of the reference motion.

### CROSS-REFERENCE TO RELATED APPLICATIONS

This application is a National Phase Entry of International Patent Application No. PCT/FR2015/053653, filed on Dec. 18, 2015, which claims priority to French Patent Application Serial No. 1462735, filed on Dec. 18, 2014, both of which are incorporated by reference herein.

### TECHNICAL FIELD

The present invention concerns a system and a method for controlling a cyclic motion of a body segment of an individual.

### BACKGROUND

The analysis and the correction of the motion of a body segment of an individual are necessary in different biomedical applications, notably rehabilitation. Thus, different pathologies are liable to alter the motions or the postures of an individual, which it is thus necessary to adapt and/or to correct. By a control of the motion or the posture, it is possible to remedy or at the very least to limit alterations due to the pathology. To this end, these motions and postures are generally carried out under the control of a physiotherapist or another specialist.

However, away from the surveillance of a therapist, the individual may once again adopt erroneous postures or motions, which adversely affect the quality and the rapidity of the rehabilitation. It would thus be desirable to provide the individual with a means for controlling his motions and postures outside of actual rehabilitation sessions and in the absence of a therapist.

With regard to the correction of posture, systems exist making it possible to compare a given posture with a reference posture and to provide, through feedback, the individual with information on the compliance of his posture [1]-[4]. However, these two solutions make it possible to study a finite number of postures but give no indication on the quality of the motion making it possible to pass from one to the other of these postures. Yet, different motions exist to arrive at a final posture, of which certain may be harmful in physiological terms. Consequently, the sole verification of a correct posture is not sufficient to verify that the motion having led to this posture has also been correctly carried out.

Furthermore, motion capture systems exist that are intended to recreate the motion of an individual on a computer, for example for animation purposes. However, these systems do not make it possible to analyse in real time the quality of the motion or to provide the individual with information on a potential incorrect motion.

Document [5] describes an item of clothing in which are integrated sensors and actuators making it possible to correct, in real time, the motions of an individual. A reference motion, corresponding to an ideal motion of a normal individual, is recorded beforehand in the system. The motion of the individual wearing the item of clothing is compared with respect to this reference motion, and, if a significant difference is measured reflecting an erroneous motion, the actuators are actuated in such a way as to correct in real time the motion of the individual. However, with such a system, the individual remains passive during the correction of the motion by the actuators, which slows his progression towards an optimised motion. Furthermore, since the reference motion is external to the individual, it remains possible that the physical capacities of the individual are not adapted to the carrying out of this ideal motion.

Document [6] describes a method for controlling a motion carried out by an individual within the scope of a rehabilitation exercise. To this end, an ideal motion is carried out beforehand by a therapist equipped with one or more three-dimensional motion sensors and recorded in the memory of a processor. The individual is next equipped with said sensors and carries out the desired motion, which is recorded and compared with the ideal motion. A feedback is provided to the individual in the form of a difference that may be visualised between the motion carried out and the ideal motion. However, the comparison is only made once the motion has been carried out completely. Consequently, it does not enable the individual to modify the execution of the motion in order to correct it from the moment that a significant difference compared to the ideal motion occurs, while being carried out. Moreover, this method is intended for the monitoring of rehabilitation exercises or sports training performed by the individual, such that it is only implemented at specific moments in the life of the individual. Yet, in order to increase monitoring efficiency, it is desirable to be able to control a motion of an individual in his daily life, notably when it involves cyclic motions such as walking.

### SUMMARY

An aim of the invention is to overcome the drawbacks of existing systems and to enable the individual to improve in an efficient manner the carrying out of a determined cyclic motion. “Motion” of a segment is taken to mean a displacement of said segment and potentially a deformation of said segment. “Cyclic motion” is taken to mean in the present text a motion carried out by the individual in a repeatable and predictable manner, that is to say that said motion is carried out several times according to a determined rhythm. As an example, walking, running, swimming, rowing, ascending or descending stairs, assembly line work or more generally any repetitive motion or motion repeated several consecutive times are cyclic motions in the sense of the present invention. “Parameter of the motion” is taken to mean any parameter making it possible to qualify and/or to quantify the motion at a given instant while it is being carried out, on a part of the motion or the motion as a whole. It may be for example the orientation, speed, acceleration, displacement amplitude, etc. of said segment or even obviously the motion as a whole.

To this end, an aim of the invention is to enable the analysis of the cyclic motion of at least one body segment of an individual and to return to the individual real-time information on the compliance of this motion with respect to a reference motion, with a view to enabling the individual to correct his motion himself. Another aim of the invention is to take account of the specific capacities of the individual, while taking account of his progression over time, without being based on a reference external to the individual.

In accordance with the invention, a system for controlling a cyclic motion of at least one body segment of an individual is proposed, comprising:
at least one three-dimensional motion sensor,
means for attaching said sensor to said body segment of the individual,
said system being characterised in that it further comprises:
a processor coupled to said sensor and capable of being carried by the individual, in which a reference cyclic motion and tolerance limits specific to the individual concerning at least one parameter of the motion to be controlled and which change depending on the progress thereof, are stored, said processor being configured to detect said cyclic motion, integrate the measurement data from said sensor in such a way as to calculate said parameter(s) of the motion of said segment and to compare, in real time, the parameter(s) of the measured motion with the previously recorded parameter(s) of the reference motion of the individual,
a feedback device coupled to said processor, designed to provide the individual with real-time information on the compliance of the parameter(s) of the measured motion with respect to the parameter(s) of said reference motion.

According to an embodiment, the means for attaching the sensor to the body segment is an item of clothing of which at least one part is adjustable to said body segment. The sensor is advantageously integral with said item of clothing. According to an embodiment, the feedback device is included in a portable apparatus distinct from the item of clothing and is connected to the item of clothing by a wireless link.

According to an embodiment, the processor is integral with the item of clothing. Alternatively, the processor is distinct from the item of clothing.

The feedback device may be configured to emit an audible signal, a visual signal and/or a tactile signal. To this end, the feedback device may comprise at least one vibrator, one electrode, one loudspeaker, and/or one screen displaying a representation of the motion.

According to a preferred embodiment, the three-dimensional sensor is an inertial unit. According to an embodiment of the invention, the system comprises at least two three-dimensional sensors intended to be made integral with a same body segment, the processor being configured to fuse the measurement data from said sensors.

The processor may be coupled to the sensor by a wireless link. In a particularly advantageous manner, the processor is configured, from a plurality of cycles of the motion detected, to calculate the average duration of a cycle and to calibrate the reference cyclic motion recorded with said average duration. According to an embodiment of the invention, the processor is configured, after carrying out a plurality of cycles compliant with the reference motion, to reduce the margin of tolerance and/or adjust the reference cyclic motion.

The invention also concerns a method for controlling the cyclic motion of at least one body segment of an individual, comprising the steps consisting in:
attaching to said segment a three-dimensional motion sensor,
carrying by the individual a processor in which a reference motion and tolerance limits concerning at least one parameter of the motion to be controlled, specific to the individual and which change depending on the progress thereof, have been previously recorded,
from the measurement data from said sensor, calculating, by means of said processor, at least one parameter of the motion of said segment,
from the motion recorded, calculating said parameter(s) of the motion to be controlled,
comparing, in real time, by means of said processor, the parameter(s) of said measured motion with the respective parameter(s) of said reference motion of the individual,
providing the individual, by means of a feedback device, with information relative to the compliance of the parameter(s) of said measured motion with respect to the respective parameter(s) of the reference motion.

### BRIEF DESCRIPTION OF THE DRAWINGS

Other characteristics and advantages of the invention will become clear from the detailed description that follows, with reference to the appended drawings in which:

FIGS. 1A to 1C illustrate different steps of implementation of the control of a cyclic motion in an individual;

FIGS. 2A and 2B are respectively a front and back view of an item of clothing intended to cover the upper part of the body of an individual, according to an embodiment of the invention; and

FIGS. 3A and 3B are respectively a front view and a back view of an item of clothing intended to cover the lower part of the body of an individual, according to an embodiment of the invention.

### DETAILED DESCRIPTION

The system for controlling a cyclic motion of at least one body segment of an individual comprises at least one three-dimensional sensor intended to measure the motions of the body segment to study. The system further comprises means for attaching each sensor to each body segment considered. Any suitable means for attachment may be employed, such as an adhesive, an adhesive tape, or instead an item of clothing intended to be worn by the individual and of which a part is intended to be adjusted closely to the segment to control. “Item of clothing” is taken to mean in the present text any piece of clothing capable of covering at least one part of the body of the individual: the limbs, including the ends thereof (feet, hands), the trunk, the head.

To this end, the item of clothing is advantageously made of an extensible textile material (fabric, knitwear, non-woven, etc.). The nature of the fibres of the textile material is determined by those skilled in the art depending on the application. Alternatively or in a complementary manner, the item of clothing may also comprise means of attachment with respect to the segment considered, such as shoe laces in the case where the item of clothing is a shoe, for example.

The three-dimensional sensor is situated in a region of the item of clothing intended to adjust itself to said segment when the item of clothing is worn by the individual. Thus, the sensor is made integral with said segment, without it being necessary to employ a means for direct attachment (for example adhesive) to said segment. The sensor is thus always positioned at the same spot of the segment even after several dressings and undressings. “Three-dimensional sensor” is taken to mean in the present text a sensor suited to providing information concerning the position and the orientation in space of a body segment to which it is attached.

Advantageously, an inertial unit is used as three-dimensional sensor, but other sensor technologies may be envisaged, such as conductive elastomers and optical fibres. In fact, sensors using conductive elastomers [7] or optical fibres [8] have already been used to measure articular angles. Elastomers have piezoresistive properties, that is to say that their deformation modifies the electric current that passes through them and optical fibres have a curvature proportional to the flow of light passing through them. By using a network of optical fibres or elastomers it is thus possible to obtain a 3D orientation of one segment with respect to another.

Advantageously, several sensors integral with a same segment make it possible to take account more precisely of the motion of said segment, notably when the motion to carry out is complex; however, in certain cases, the use of a single sensor for a segment may be sufficient, for example when the motion to perform is not very variable or is carried out in a plane. Furthermore, the system may comprise sensors intended to be made integral with different segments of the body when the motion involves several segments. For example, to control the motion of a limb, sensors are integrated with different segments composing the limb, and potentially at least one sensor integral with the trunk of the individual, are integrated with an item of clothing.

The system furthermore comprises at least one processor coupled to the sensor—or to the plurality of sensors if needs be—the link being able to be wired or wireless. The processor is integral with the individual, for example when said processor is arranged in or on an item of clothing. Alternatively, the processor of a smartphone or any other portable device capable of being carried by the individual may be used to perform the calculations.

The processor is configured to record the motion of the individual by integrating the measurement data of the three-dimensional sensor(s) and to compare it, in real time, with a reference motion. To this end, the processor implements a comparison of signals (namely one or more signals representative of the motion being carried out and one or more signals representative of the reference motion). The algorithms for comparing signals point by point between extreme limits are conventional in themselves and will not be described in detail herein. This comparison is made in real time in order to take account of the dynamic aspects of the motion and its temporal organisation.

When several sensors are used, the processor implements a data fusion algorithm to take account of the measurement data from the different sensors. In a particularly advantageous manner, said reference motion is not defined a priori, independently of the individual, but is on the contrary recorded beforehand on the individual by means of sensors, under the control of a therapist or another specialist. Thus, the reference motion depends on the individual and his motor capacities; it does not correspond to an ideal motion in absolute terms but to a motion considered as optimal for the individual in question.

To record this reference motion in the memory of the system, the individual equipped with the sensors performs the motion several times under the control of the therapist, who records several motions that he considers optimal and which will enable the most appropriate reference for the patient to be calculated. All of these recorded motions also make it possible to calculate the dynamic execution limits concerning all of the motion parameters to be controlled within which the motion will be considered correct. The therapist may also decide to set these limits a priori. For a same optimal motion, different limits may exist having decreasing amplitudes corresponding to an increase in the difficulty of the exercise as the individual progresses.

It will be noted that the specialist may defined the margin of error for a particular parameter of the motion, according to the improvement objective given to the individual. For example, in the case of walking, such a parameter may be: the speed of carrying out the motion, the amplitude of the motion, the rhythm of posing a heel on the ground, the symmetry (non-limiting list). Thus, the feedback could be triggered specifically in the event of a difference compared to said parameter and not necessarily with respect to the motion considered as a whole.

In addition, the system is capable of conserving the history of a defined number of final tests, which enables the limits set beforehand around the optimal motion to change automatically over time depending on the progress made by the individual. The system is thus made adaptive to the change in the motion of the individual. Naturally, as the training of the individual progresses and as his capacities change, the therapist may also be brought to modify the optimal motion and/or to refine the limits around the optimal motion.

Since the processor is integral with the individual (for example integrated in an item of clothing or integrated in a smartphone carried by the individual), the individual benefits from full freedom in his motions, since he does not need to be connected to a computer which would perform the recording or the comparison of motions. Advantageously, the link between the sensors and the processor is made wireless in order to conserve this freedom of motion. Since the motion is cyclic, the system is automatically capable of recognising the motion to control when the latter is repeated a certain number of consecutive times, and becomes inactive again from the moment where the user changes activity. It is obviously understood that several cyclic motions may be stored and the system will then be capable of recognising them in order to use the corresponding reference motion.

The system further comprises at least one feedback device coupled to the processor and intended to supply the individual with information on the compliance of the motion with respect to the reference motion. This information may be supplied in the form of a visual, audible and/or tactile signal. For example, dynamic limits being defined beforehand, the motion is considered as correct (that is to say compliant with the reference motion) if the result of all of the comparisons between the parameters of the reference motion and the parameters of the motion being carried out is within said limits. Conversely, if the result of the comparison is outside these limits for a duration defined beforehand, the processor transmits to the feedback device an order to emit a signal which may be different depending on the nature of the calculated error.

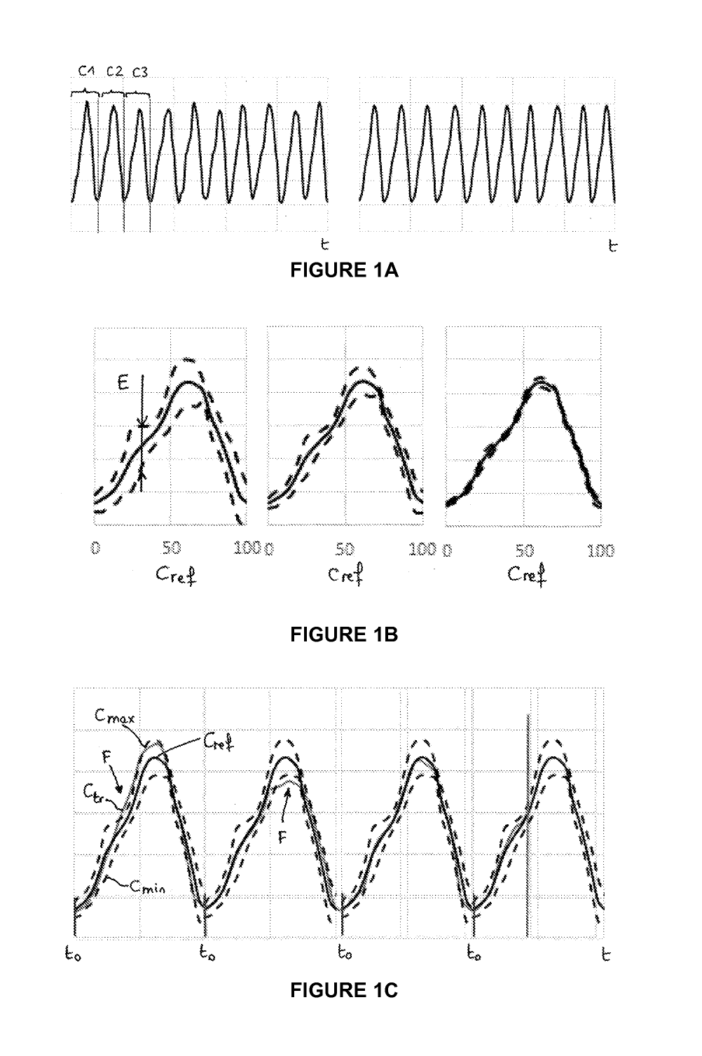

FIG. 1A illustrates the recording of data of a determined cyclic motion. In this step, the individual is equipped with the three-dimensional motion sensor(s). Under the control of a specialist (for example a therapist, an ergonomist), the individual carries out the cyclic motion to the best of his current capacities. A motion considered as acceptable is thus recorded then the parameter(s) of the motion to be controlled are chosen (the left curve representing such a parameter depending on time t), each cycle being designated by the reference C₁, C₂, C₃, etc. The right curve represents the ideal data of this motion parameter, the motion being able to be carried out either by another healthy individual, or by the individual himself at a later stage of his treatment, reflecting a progression in accomplishing the motion. Potentially, a certain number of intermediate recordings (not represented) may exist as the individual progresses.

FIG. 1B illustrates the calculation of a reference cycle C_{ref} from the recording represented in FIG. 1A, corresponding to an arithmetic mean of the duration of a certain number of cycles, as well the calculation of the error E accepted for said cycle C_{ref}. In the example illustrated in FIG. 1B, three levels of difficulty exist corresponding to a progressively decreasing margin of error: the left curve illustrates a high margin of error, corresponding to an easy exercise, the right curve illustrates a minimum margin of error, corresponding to a difficult exercise, and the middle curve illustrates an intermediate margin of error, corresponding to an exercise of moderate difficulty. Here, the margin of error is represented by dotted lines on the whole of the motion (that is to say between 0 and 100% of the cycle), but said motion may only be defined at an instant, or also only on a part of the motion.

The processor may firstly apply the highest margin then, once the motion of the individual is compliant with said margin, the processor applies the intermediate margin of error as long as the motion of the individual is not compliant with said margin and, finally, the processor applies the minimum margin of error. Once the individual is capable of reproducing the acceptable motion while respecting said minimum margin of error concerning the parameter(s) of the motion which are controlled, the motion is adjusted so as to tend towards the ideal motion, the margins of error defined previously being applied to this adjusted motion.

FIG. 1C schematically illustrates the implementation of the method in the daily life of the individual. The individual is equipped with the three-dimensional motion sensor(s) that are adjusted on the body segment(s) concerned and which, thanks to the item of clothing which makes it possible to make them integral with the individual, are virtually invisible for third parties.

When the individual begins to perform a recorded cyclic motion, the processor uses the first cycles to detect said motion and to calculate the average duration of the cycle. In fact, it is considered that a given cyclic motion always has a same organisation over time. Consequently, even if the motion is carried out at a speed different to that of the recorded motion, the organisation of this motion remains identical and it suffices that the processor applies the measured average duration to calibrate the recorded reference cycle.

For example, when it involves the walking motion, this is constituted of two main phases:

(I) the support phase, which corresponds to the period where at least one part of the foot is in contact with the ground, which typically extends between 0 and 60% of the gait cycle, and which is broken down into three secondary phases:
(I.1) the taligrade phase, which starts with the initial contact of the heel with the ground and continues with the loading of the lower right limb; said phase extends between 0 and 10% of the gait cycle;
(I.2) the plantigrade phase, which starts when the foot rests on the sole of the foot and ends when the heel loses contact with the ground; said phase extends between 10 and 50% of the gait cycle;
(I.3) the digitigrade phase, which starts when the heel is lifted and ends when the foot has been taken off the ground; said phase extends between 50 and 60% of the gait cycle;

(II) the oscillating phase where the foot is no longer in contact with the ground and which enables the lower limb to advance, which extends between 60 and 100% of the gait cycle, and which is divided into two secondary phases:
(II.1) the phase of shortening the leg;
(II.2) the phase of extending the leg.
   Next, as the motion is accomplished by the individual (represented by the curve Cₜᵣ) over time t, the processor detects each start of cycle (noted t₀) and applies the predetermined margin of error (shown schematically by the curves Cₘᵢₙ and Cₘₐₓ that surround the reference cycle C_{ref}).

When the motion carried out goes beyond the margin of error, the processor triggers a feedback (noted F) to inform the individual of the error and to incite him to correct his motion. Thus, on the first cycle, a feedback F is triggered because the recorded motion Cₜᵣ exceeds the limit Cₘₐₓ; on the second cycle, a feedback F is triggered because the recorded motion Cₜᵣ passes beyond the limit Cₘᵢₙ, whereas no feedback is triggered on the third cycle because the recorded motion remains within the interval [Cₘᵢₙ, Cₘₐₓ]. As may be seen in FIG. 1C, the feedback is triggered in real time, that is to say from the moment that an overrun of the margin of error is measured for the parameter(s) of the motion considered. Thus, the individual can modify the carrying out of the motion in real time, from the cycle considered, without waiting to have finished a complete exercise.

Preferably, the feedback device may be made integral with the individual by any appropriate means. Such is the case notably when the feedback signal is tactile. For example, the feedback device comprises at least one electrode or one vibrator maintained in contact with the skin of the individual. If the sensor(s) and potentially the processor are integrated in an item of clothing, the feedback device may also be integrated in said item of clothing.

A single feedback device may be sufficient to provide the individual with information according to which the motion is compliant or not with the reference motion. Potentially, several feedback devices may be used and spread out so as to provide the individual with more precise information on the portion of motion not compliant with the reference motion, the segment not having carried out a motion compliant with the reference motion or on the parameter of the motion that is not compliant. Potentially, the intensity of the tactile signal may be modulated depending on the error recorded.

In the case of a visual signal, the feedback device may for example comprise a light that lights up when the result of the comparison is outside of the predefined limits. Said light may be made integral with the individual by any suitable means, such as an item of clothing as mentioned above. Alternatively, the feedback device may comprise the screen of a smartphone, which displays visual information. In the case of an audible signal, the feedback device may comprise a loudspeaker. Said loudspeaker may be integral or not with the individual.

Alternatively, the feedback device may be included in an apparatus distinct from the item of clothing, preferably a portable apparatus such as a mobile telephone or a mobile personal assistant. In this case, the feedback device is coupled to the processor by a wireless technology, according to an appropriate communication protocol. The feedback may for example consist in symbolising the segment(s) involved in the motion and displaying in a different colour the segment(s) in question when the motion is not compliant with the reference motion on the screen of the apparatus. Alternatively or in a complementary manner, the feedback may also consist in a vibration of the apparatus signalling a non-compliance of the motion with respect to the reference motion. This enables in particular a feedback perceptible uniquely by the individual and not by the people around him.

The feedback may be carried out in real time, for example in the case of a tactile device, to enable the individual to take account of the non-compliance of the motion as soon as it is recorded. Alternatively, the comparison of the recorded motion and the reference motion may be memorised so as to be able to be played back later.

In all cases, the feedback aims uniquely to provide the individual with information in order to lead said individual to correct his motion himself, and not to correct by actuators the motion carried out by the individual. Thus, the individual is fully active in the approach for correcting the motion, which is thus more efficient. On the other hand, the fact that the reference motion is specific to the individual, while taking account of his initial physical capacities and their change, and not external thereto (as would be a standard ideal motion) enables the individual to attain the set objective more easily and makes this learning more efficient.

Furthermore, the processor can memorise the history of successive implementations of the motion, to represent the failures and successes of the individual during his learning. In this respect, the case where the sensor(s) are integral with an item of clothing is advantageous in so far as this embodiment ensures a constant emplacement of each sensor with respect to a segment, such that the different tests are repeatable.

The system further comprises a battery enabling the electrical supply of the sensor(s), the processor and, if needs be, the feedback device(s). To minimise the necessity of recharging or replacing the battery, elements are preferably employed for which the electrical consumption is minimal.

The integration of the different elements in the item of clothing may be achieved by any appropriate means for making integral, for example by bonding onto the internal or external surface of the item of clothing, by incorporation in the fibres of the textile during the manufacture thereof, by sewing, etc. On account of the flexibility of the textile material of the item of clothing, this does not hinder the gestures of the individual and thus has a certain comfort of use. In addition, since the item of clothing has a small thickness and hugs the shape of the body, it is relatively discrete and may potentially be worn under another item of clothing, such that persons other than the individual do not perceive its technical function. To favour the comfort of the item of clothing, the different elements are advantageously miniaturised in such a way as to have a weight and a bulk that are as low as possible.

Depending on the pathology and/or the individual, erroneous postures or motions may involve different sensorial systems, mainly the visual system, the proprioceptive system and/or the vestibular system. To identify the system concerned in the non-compliance of the motion, the sensors are advantageously chosen and arranged in the item of clothing to make it possible to determine the contribution of each system to the motion.

Thus, the contribution of the proprioceptive system may be estimated by comparing the position and/or the orientation of a segment during the motion compared to the reference motion. To this end, three-dimensional sensors positioned on the particular segments that it is wished to evaluate the proprioception are used.

The contribution of the vestibular system may be estimated by evaluating the equilibrium of the individual with regard to the linear or angular acceleration of one or more sensors. To this end, at least one three-dimensional sensor or simply an accelerometer situated on the head is used. It is possible to refine this measurement with other sensors situated for example on the pelvis or the upper part of the body to assess the general equilibrium of the subject.

The contribution of the visual system may be estimated by evaluating the position of the head with respect to the shoulders. To this end, a three-dimensional sensor situated on the head and another situated on a shoulder or in the upper part of the back are used. Depending on the system identified as faulty when carrying out the motion, the feedback device(s) may be arranged in such a way as to indicate to the user the nature of the error and its location.

The invention may find applications in numerous fields. Among these may be cited notably rehabilitation or ergonomics. Outside of the medical field, the sports field may be cited and more generally all learning situations which involve motricity and the repetition of gestures (training of the individual aiming to improve a gesture).

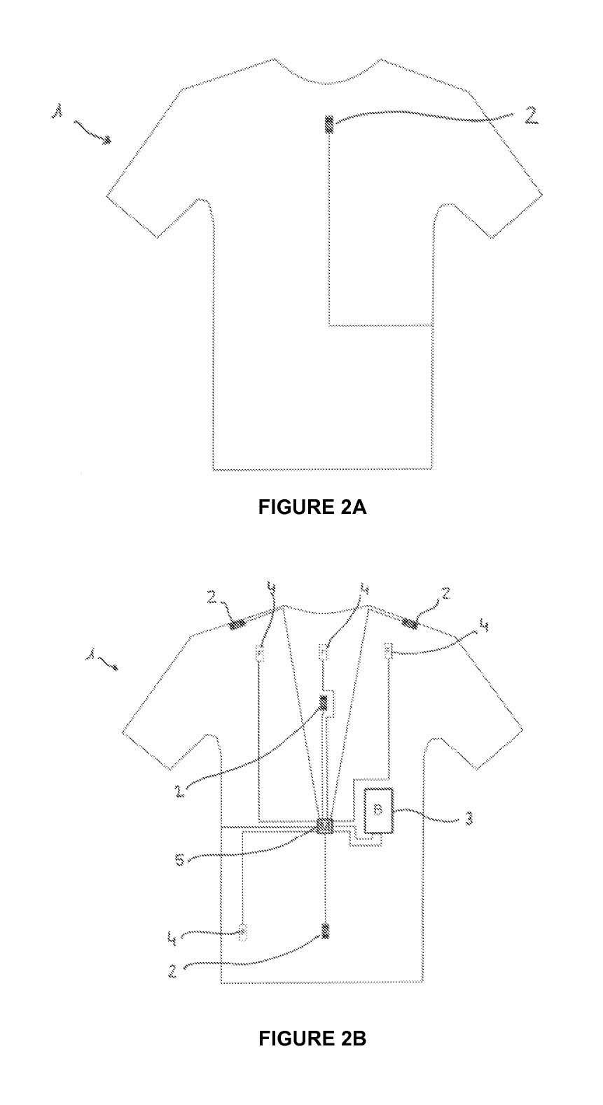

FIGS. 2A and 2B illustrate an embodiment of the invention, respectively in front view and in back view. In this example, the item of clothing 1 is a T-shirt made of an extensible textile material, so as to be able to be closely adjusted to the upper part of the body of an individual. The item of clothing comprises several sensors 2, namely:
three sensors in the sagittal plane: a sensor on the front of the item of clothing, near to the neck, and two sensors on the back of the item of clothing, respectively in the upper and lower part of the back;
two sensors each arranged on a shoulder in the back of the item of clothing, in a symmetric manner with respect to the sagittal plane. In this case, the body segment of which the motion is studied is the trunk of the individual.
   In this case, the sensors are inertial units arranged in such a way as to take account of the motions of the chest of the individual in three dimensions.

The item of clothing 1 further comprises four feedback devices 4 (for example electrodes or vibrators): three of these devices are arranged in a plane horizontal to the back of the item of clothing, and one is arranged at the bottom of the back, at a distance from the sagittal plane. The item of clothing 1 furthermore comprises a processor 5 coupled both to the sensors 2 and to the feedback devices 4. Potentially, if there are a large number of sensors, the item of clothing may comprise several processors. Alternatively, the processor could be distinct from the item of clothing and belong, for example, to a smartphone carried by the individual. In this example, the links between the processor, the sensors and the feedback devices are represented in wired form, but they could potentially be made in wireless form by means of a suitable communication protocol.

Finally, the item of clothing 1 further comprises a battery 3 intended to supply the processor 5, the sensors 2 and the feedback devices 4 with energy. The sensors, the feedback devices, the processor and the battery may be made integral with the item of clothing by any means. Among the means for making integral may be cited bonding, sewing, integration in the method of manufacture (for example weaving) of the textile material, etc.

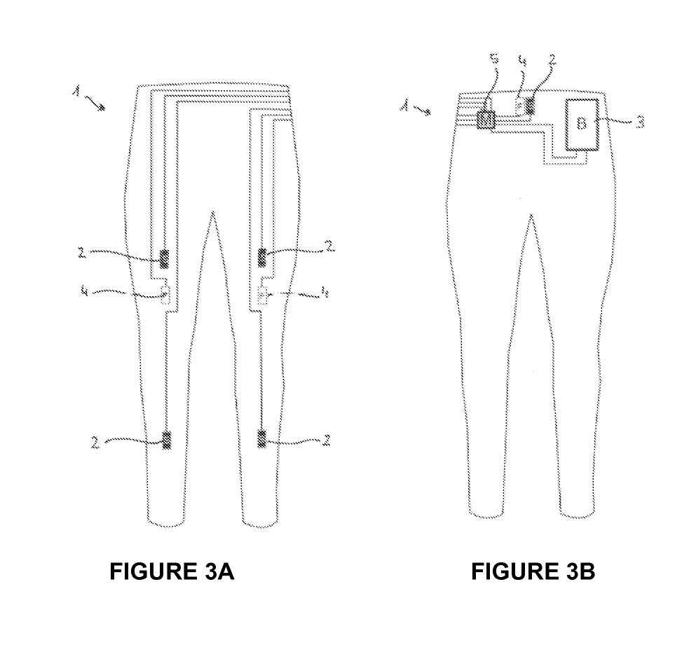

FIGS. 3A and 3B illustrate another embodiment of the invention, respectively in front view and in back view. In this example, the item of clothing 1 is a pair of trousers made of an extensible textile material, so as to be able to be closely adjusted to the lower part of the body of an individual. The reference signs reproduced from FIG. 1 correspond to the same elements.

In this case, the item of clothing 1 comprises two sensors on the front of each leg, respectively above the knee and just below the knee, and a sensor situated at the back, near to the waist. The segments of interest are the lower and upper parts of each leg. The item of clothing 1 further comprises a feedback device 4 situated on the front of each knee. The processor 5 and the battery 3 are arranged in the back of the item of clothing, near to the waist.

Finally, it goes without saying that the examples that have been given are only particular illustrations that are in no way limiting as regards the application fields of the invention.

### REFERENCES

[1] WO 2009/112281
[2] US 2011/0063114
[3] US 2013/0184611
[4] US 2013/0207889
[5] US 2014/303529
[6] WO 2008/129442
[7] Williams M J, Haq I, Raymond Y L, Dynamic measurement of lumbar curvature using fibre-optic sensors, Medical Engineering and Physics, 2010.
[8] Tognetti A, Lorussi F, Dalle Mura G, Carbonaro N, Pacelli M, Paradiso R, De Rossi D, New generation of wearable goniometers for motion capture systems, Journal of NeuroEngineering and Rehabilitation, 2014.

1. A system for controlling a cyclic motion of at least one body segment of an individual, comprising:
at least one three-dimensional motion sensor;
an attachment attaching the sensor to the body segment of the individual;
a processor coupled to the sensor and capable of being carried by the individual, in which a reference cyclic motion and tolerance limits specific to the individual concerning at least one parameter of the motion to be controlled and which change depending on the progress thereof, are stored;
the processor being configured to detect the cyclic motion, integrate the measurement data from the sensor in such a way as to calculate the parameter(s) of the motion of the segment and to compare, in real time, at least one parameter of the measured motion with a parameter of the previously recorded reference motion of the individual; and
a feedback device coupled to the processor, adapted to provide the individual with real-time information on compliance of the parameters(s) of the measured motion with respect to the parameter(s) of the reference motion.

2. The system according to claim 1, wherein the attachment is an item of clothing of which at least one part is adjustable to the body segment, the sensor being integral with the item of clothing.

3. The system according to claim 2, wherein the feedback device is included in a portable apparatus distinct from the item of clothing and is connected to the item of clothing by a wireless link.

4. The system according to claim 2, wherein the processor is integral with the item of clothing.

5. The system according to claim 2, wherein the processor is distinct from the item of clothing.

6. The system according to claim 1, wherein the feedback device is configured to emit an audible signal.

7. The system according to claim 1, wherein the feedback device is configured to emit a visual signal.

8. The system according to claim 1, wherein the feedback device is configured to emit a tactile signal.

9. The system according to claim 1, wherein the feedback device comprises at least one of: a vibrator, an electrode, a loudspeaker, and a screen displaying a representation of the motion.

10. The system according to claim 1, wherein the three-dimensional sensor is an inertial unit.

11. The system according to claim 1, comprising at least two three-dimensional sensors intended to be made integral with a same body segment, the processor being configured to fuse the measurement data from the sensors.

12. The system according to claim 1, wherein the processor is coupled to the sensor by a wireless link.

13. The system according to claim 1, wherein the processor is configured, from a plurality of cycles of the motion detected, to calculate an average duration of a cycle and to calibrate the reference cyclic motion recorded with the average duration.

14. The system according to claim 1, wherein the processor is configured, after execution of a plurality of cycles compliant with the reference motion, to reduce a margin of tolerance and/or adjust the reference cyclic motion.

15. A method for controlling cyclic motion of at least one body segment of an individual, comprising:
attaching to said segment a three-dimensional motion sensor;
carrying by the individual a processor in which a reference motion and tolerance limits concerning at least one parameter of the motion to be controlled, specific to the individual and which change depending on the progress thereof, have been previously recorded;
calculating at least one parameter of the motion of the segment from measurement data of the sensor, by the processor;
calculating said at least one parameter of the motion to be controlled from the motion recorded;
comparing in real time, by the processor, the at least one parameter of the measured motion with at least one parameter of the reference motion of the individual; and
providing feedback to the individual with information relative to the compliance of the at least one parameter of the measured motion with respect to the at least one parameter of the reference motion.
